(19) United States
(12) Patent Application Publication
(10) Pub. No.: US 2023/0024494 A1
(43) Pub. Date: Jan. 26, 2023

(54) NEW SALIVA-BASED LATERAL-FLOW ANTIBODY TEST PLATFORM FOR ASSESSING INFECTIONS AND VACCINATION EFFICACY

(71) Applicant: Nirmidas Biotech, Inc., Palo Alto, CA (US)

(72) Inventors: Meijie Tang, Cupertino, CA (US); Dingying Shan, Foster City, CA (US); Hongjie Dai, Cupertino, CA (US)

(21) Appl. No.: 17/827,451

(22) Filed: May 27, 2022

Related U.S. Application Data
(60) Provisional application No. 63/208,443, filed on Jun. 8, 2021.

(51) Int. Cl. G01N 33/569; G01N 33/543; G01N 21/64

(52) U.S. Cl. CPC G01N 33/56983; G01N 33/54388; G01N 21/6428; G01N 2021/6439

(57) ABSTRACT

Sensitive detection of IgG antibodies against SARS-CoV-2 is important to assessing immune responses to viral infection or vaccination and immunity duration. Antibody assays using non-invasive body fluids such as saliva could facilitate mass testing including young children, elderly and those who resist blood draws, and easily allowing longitudinal testing/monitoring of antibodies over time. Here, we developed a new lateral flow (nLF) assay that sensitively detects SARS-CoV-2 IgG antibodies in the saliva samples of vaccinated individuals and previous COVID-19 patients. The 25 minutes nLF assay detected anti-spike protein (anti-S1) IgG in saliva samples with 100% specificity and high sensitivity from both vaccinated (99.51% for samples ≥19 days post 1st Pfizer or Moderna mRNA vaccine dose) and infected individuals. Antibodies against nucleocapsid protein (anti-NCP) was detected only in the saliva samples of COVID-19 patients and not in vaccinated samples, allowing facile differentiation of vaccination from infection. Salivary SARS-CoV-2 anti-S1 IgG antibodies correlated with that in matched dried blood spot (DBS) samples measured by a quantitative pGOLD™ lab-test, showing similar evolution trends post vaccination. The new salivary rapid test platform is applicable to non-invasive detection of antibodies against infection and vaccination for a wide range of diseases.

### CROSS-REFERENCE TO RELATED APPLICATIONS

This application claims priority to, and the benefit of, U.S. Provisional Patent Application No. 63/208,443, filed Jun. 8, 2021, the entire contents of which are herein incorporated by reference in its entirety for all purposes.

### FIELD

The present disclosure concerns lateral flow assays for antibody detection.

### BACKGROUND

The COVID-19 pandemic, due to severe acute respiratory syndrome coronavirus 2 (SARS-CoV-2), has been presenting a major global health crisis [1-6]. It is clear that mass vaccination offers a viable path back to normalcy [7, 8]. As of May 2021, 17 different COVID-19 vaccines across four platforms are rolled out across the globe, with more than 300 additional vaccine candidates still in development. Over 1891 million vaccine doses have been administered worldwide, raising the hope of emerging from the pandemic [9]. Questions linger on the duration of the COVID-19 protection offered by vaccination. Since antibody response and persistence vary among individuals and vaccines, antibody assessment at the individual level could help to evaluate antibody persistence and the necessity of booster doses. Various antibody tests have been developed using enzyme-linked immunosorbent assay (ELISA) [10], nanostructured plasmonic gold (pGOLD) assay [11], chemiluminescence (CLIA) [12] and lateral flow (LF) technologies [13]. Importantly, antibodies against spike proteins have shown high correlation with neutralizing antibodies for COVID-19, offering a simple, first-line assessment of immunity without resorting to assays involving live viruses [14, 15]. Currently most SARS-CoV-2 antibody tests require invasive blood/serum processes and professional operations, limiting the accessibility and frequency of testing. To facilitate personalized monitoring of SARS-CoV-2 antibody persistence, non-invasive and easy testing strategies are desired. Saliva represents an appealing biofluid for non-invasive SARS-CoV-2 antibody detection, with the ease of self-collection and is pain-free [16-18]. Previous work has shown that saliva's IgG antibody profile is similar to that of blood for various infectious diseases including SARS-CoV-2 [11, 19-22]. However, the main drawback of testing saliva samples is its much lower concentration of antibodies than in serum, plasma and blood, demanding high analytical sensitivity of detection methods [23]. The high viscosity of saliva samples also presents a challenge to flow-based rapid testing. No saliva-based antibody LF assay exist currently for SARS-CoV-2 vaccination or infection assessment at the point-of-care (POC).

### SUMMARY

Set forth herein is a new lateral flow (nLF) test highly sensitive for SARS-CoV-2 IgG antibody detection in saliva. Conventional antibody LF assays mostly flow a biofluid sample containing targeting antibodies through a conjugate pad containing gold-antigen complexes to form gold-antigen-antibody (Au-antigen-antibody) complexes that migrate towards the detection zone and bind to the test line containing immobilized anti-human antibodies. A visually readable test line due to the bound Au nanoparticles suggests a positive test result [24]. A limitation of this LF approach is that biofluid samples always contain abundant non-specific human antibodies that compete with the specific antibodies captured by the gold-antigen (Au-antigen) complex for the anti-human antibody binding sites on the test line, causing low sensitivity and non-linearity of the assay. Further, saliva samples are viscous, requiring high degrees of dilution in order to run on conventional LF strips, further lowering the analytical sensitivity of saliva LF tests.

Set forth herein are new Au nanoparticle-antigen (or magnetic Fe₃O₄ core/Au shell nanoparticle-antigen) complexes, then when incubated with a saliva sample in a reaction tube to capture antibodies specific to the antigen, and includes removing the non-specific antibodies (by micro-centrifuging or magnetic purification), and then applying the Au-antigen-antibody complexes to a LF test card containing anti-human IgG signal line for detection. The entire assay time was about 25 min. A portable, economic micro-centrifuge or magnetic suction were employed for removing the non-specific antibodies, making the assay user-friendly in POC settings. The nLF assay affords high anti-SARS-CoV-2 S1 IgG sensitivity (>99%) with vaccinated saliva samples collected ≥19 days post first Pfizer or Moderna mRNA vaccine dose accompanied by 100% specificity. By applying both SARS-CoV-2 spike protein S1 subunit and nucleocapsid (also known as nucleoprotein, NCP) to make Au-antigen complexes, Au-S1 and Au-NCP for nLF assays, the instant disclosure differentiated saliva samples from SARS-CoV-2 vaccinated subjects from COVID-19 infected subjects. Also, anti-S1 antibodies in saliva showed a similar trend as those measured by a quantitative serology test performed on the pGOLD platform [11]. Overall, the rapid nLF assay platform herein can be easily implemented at various POC sites including clinical laboratories, pharmacies, or eventually at home for non-invasive, highly accurate SARS-CoV-2 IgG monitoring.

Conventional antibody LF assays (such as e.g., FDA EUA Nirmidas MidaSpot™ COVID-19 Ab test) typically flow an antibody containing biofluid sample through a conjugate pad pre-deposited with gold-antigen complexes to form gold-antigen-antibody (Au-antigen-antibody) complexes that migrate towards the detection zone and bind to the test line containing immobilized anti-human antibodies. A visually readable test line due to the bound Au nanoparticles suggests a positive test result. A limitation of this LF approach is that biofluid samples always contain abundant non-specific human antibodies that compete with the specific antibodies captured by the gold-antigen (Au-antigen) complex for the anti-human antibody binding sites on the test line, causing low sensitivity and non-linearity of the assay. Further, some types of body fluid are difficult to use in conventional assays. For example saliva samples are viscous, requiring high degrees of dilution in order to run on conventional LF strips, further lowering the analytical sensitivity of saliva LF tests. For these reasons no saliva-based antibody LF tests exist for SARS-CoV-2 infection or vaccination assessment.

Accordingly, the instant disclosure sets forth a new LF approach including an Au nanoparticle-antigen (or magnetic core/Au nanoparticle-antigen) complexes, then incubated with a saliva sample in a reaction tube to capture antibodies specific to the antigen, removed the non-specific antibodies (by micro-centrifuging or magnetic purification), and then applied the Au-antigen-antibody complexes to a LF test card containing anti-human IgG signal line for detection. The step of removing unbound non-specific antibodies by micro-centrifuging or magnetic purification prevented competing antibodies in the sample from binding to the test line, drastically enhancing the detection sensitivity of specific antibodies over the traditional LF assay. The new LF method also avoided the high viscosity problem of saliva samples. A portable, economic micro-centrifuge or Fe₃O₄ core/Au shell particles/magnetic suction were employed for removing the non-specific antibodies, making the assay user-friendly in POC settings.

The nLF antibody test achieves at least 50 times lower limit of detection than conventional antibody LF test such as MidaSpot™. The nLF antibody test can give quantitative antibody concentration with much improved linearity as compared to conventional antibody LF tests.

The nLF assay affords high anti-SARS-CoV-2 S1 IgG sensitivity (>99%) with vaccinated saliva samples collected ≥19 days post first Pfizer or Moderna mRNA vaccine dose accompanied by 100% specificity. By applying both SARS-CoV-2 spike protein S1 subunit and nucleocapsid (also known as nucleoprotein, NCP) to make Au-antigen complexes, Au-S1 and Au-NCP for nLF assays, we differentiated saliva samples from SARS-CoV-2 vaccinated subjects from COVID-19 infected subjects.

Another novel feature is that is that the new LF platform allows antibody avidity measurement using saliva samples. To the best of the inventor's knowledge, this is the first time saliva samples are used for antibody avidity test.

Other features objects and advantages will be apparent from the disclosure that follows.

### BRIEF DESCRIPTIONS OF THE DRAWINGS

FIG. 1A shows Au-antigen-IgG complex formation.

FIG. 1B shows a lateral flow test card.

FIG. 1C shows an operation of the test card.

FIG. 2A shows a plot of Saliva anti-S1-IgG Signal as a function of concentration (m/mL).

FIG. 2B shows an operation of the test card.

FIG. 2C shows a plot of plot of Saliva IgG Signal as a function of Vaccinated D42 Saliva and also Infected D81 Saliva.

FIG. 3A shows a plot of plot of Saliva IgG Signal for twenty trials of Vaccinated Saliva (D42-55).

FIG. 3B shows a plot of plot of Saliva IgG Signal for twenty-one trials of Infected Saliva.

FIG. 4A shows a plot of anti-S1 IgG Positive Rate (%) as a function of Days post 1^{st} Vaccine Dose.

FIG. 4B shows a plot (top) of Salivary anti-S1 IgG Signal (nLF) as a function of Days post 1^{st} Vaccine Dose and also a plot (bottom).

FIG. 5A shows a plot of plot of Saliva anti-S1 IgG Signal as a function of weeks post 1^{st} Vaccine Dose.

FIG. 5B shows a plot of plot of Saliva IgG Signal as a function of Vaccination Time.

FIG. 6A shows test strips.

FIG. 6B shows test strips.

FIG. 7A shows a plot of Sensitivity as a function of Specificity.

FIG. 7B shows a plot of Sensitivity as a function of Specificity.

FIG. 8A shows images of fluorescent pGOLD.

FIG. 8B shows images of fluorescent pGOLD.

FIG. 8C shows images of fluorescent pGOLD.

FIG. 8D shows images of fluorescent pGOLD.

FIG. 9A shows a magnetic rack for removing unbound non-specific antibodies.

FIG. 9B shows a mAu-S1-IgG complex collection by magnetic force.

FIG. 9C shows results from mAu based nLF for anti-S1 IgG positive and negative saliva samples

FIG. 10 shows plots of Salivary anti-S1 IgG 1-13 as a function of weeks post vaccination in different individuals

The patent or application file contains at least one drawing executed in color. Copies of this patent or patent application publication with color drawing(s) will be provided by the Office upon request and payment of the necessary fee.

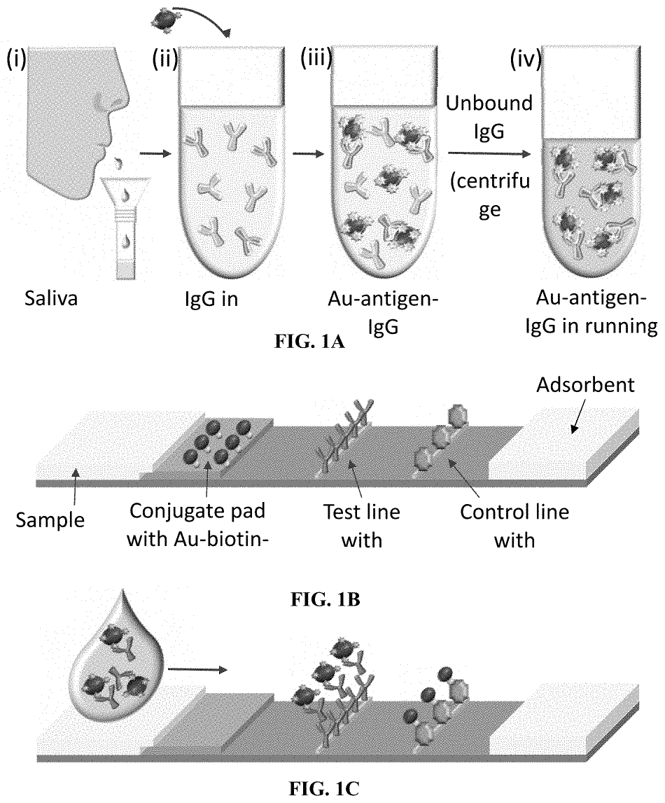

FIGS. 1A, 1B, and 1C show A new lateral flow (nLF) test for SARS-CoV-2 antibody testing in saliva. a, Au-antigen-IgG complex formation. After saliva sample collection (i), Au-antigen (antigen=S1 or NCP of SARS-CoV-2) complexes are added to incubate with the sample (ii) and capture antigen specific IgG antibodies to form Au-antigen-IgG complexes (iii). Then a micro-centrifuging or magnetic purification step is applied to collect the Au-antigen-IgG complexes and remove the unbound antibodies. The resulting Au-antigen-IgG complexes are dispersed in a running buffer before being added to a specially designed LF testing card. b, Design of the nLF testing card. The card was mainly comprised of a sample pad, a conjugate pad with pre-deposited colloidal gold nanoparticle-biotinylated BSA (Au-biotin-BSA) complexes, a test line immobilized with anti-human IgG, a control line immobilized with streptavidin, and an adsorbent pad. c, Operation of the nLF assay. When antigen-specific IgG positive samples are used, Au-antigen-IgG complexes in a running buffer flow through the test card. Au-antigen-IgG complexes are captured by anti-human IgG on the test line, producing a red-colored positive signal on the test line. Au-biotin-BSA complexes on the conjugate pad flow together with the running liquid and are captured by streptavidin on the control line, giving a red-colored control signal. While negative samples or buffer alone only produce a red line in the control line.

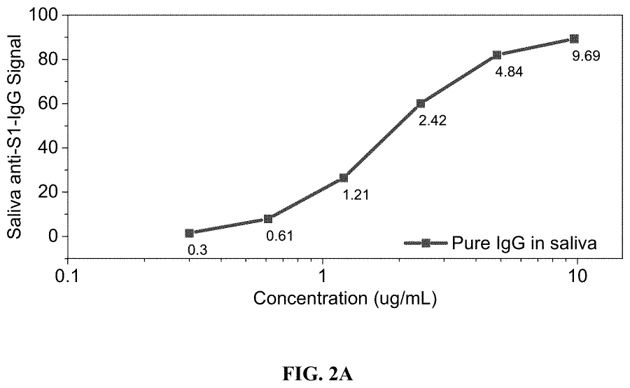

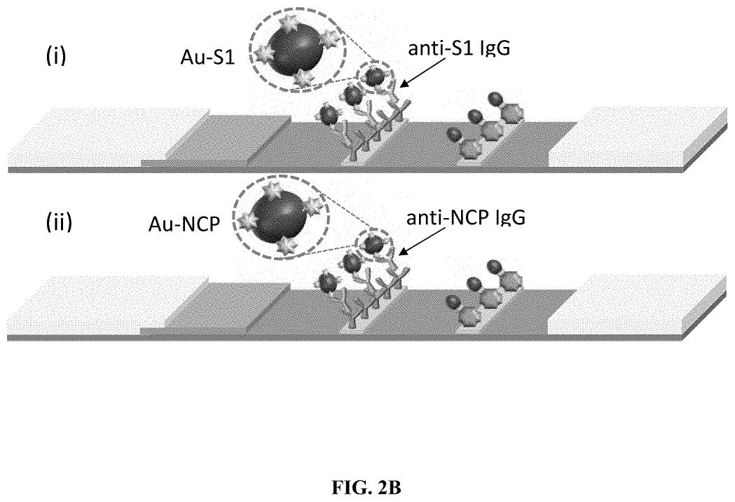

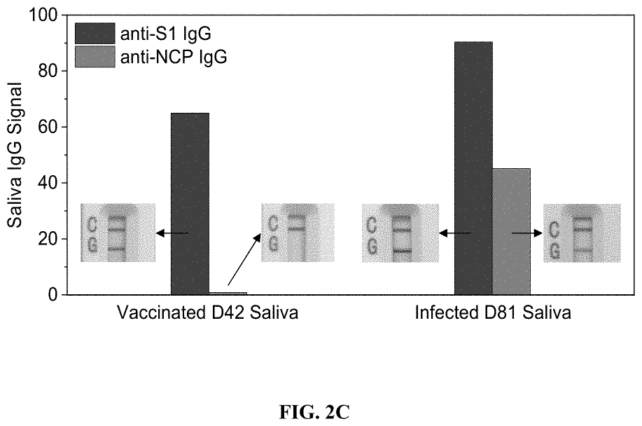

FIGS. 2A, 2B, and 2C show Highly sensitive and multifunctional nLF test for SARS-CoV-2 antibody testing in saliva. a, Detection limit of anti-SARS-CoV-2 spike protein IgG in saliva. Purified recombinant anti-SARS-CoV-2 spike protein IgG are spiked into a negative saliva sample and performed serial dilutions for establishing the nLF assay detection limit for anti-spike IgG in saliva. Based on the test card photos and quantified signal from ImageJ software, an estimated antibody detection limit of ˜0.30 μg mL-1 are obtained. b, Schematic representation of nLF for anti-S1 IgG detection (i) and anti-NCP IgG detection (ii). c, Representative photos and IgG signals obtained from nLF for vaccinated and infected saliva samples. A vaccinated saliva sample (42 days post 1st RNA vaccine dose from Moderna) shows positive anti-S1 IgG and negative anti-NCP IgG; while an infected saliva sample (81 days post symptom onset) shows both positive anti-S1 IgG and anti-NCP IgG.

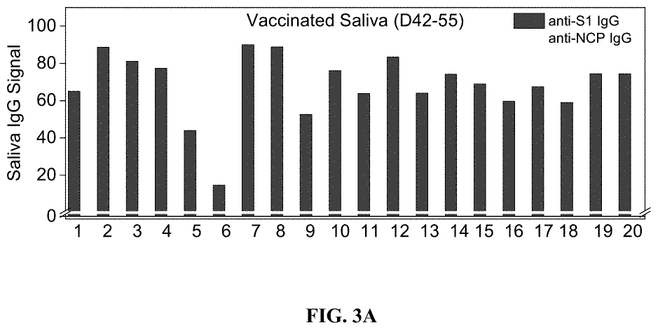

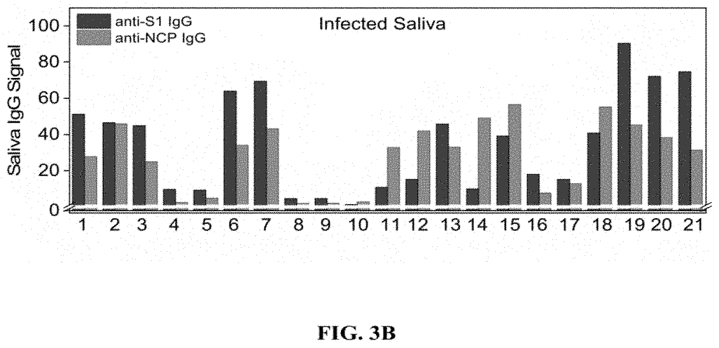

FIGS. 3A and 3B show Differentiation of vaccination and infection by the nLF test. a, Anti-S1 IgG and anti-NCP IgG signals for 20 vaccinated samples (42-55 days post 1st vaccine dose). The cutoff value of 0.921 for anti-S1 IgG signal and 1.004 for anti-NCP IgG signal. Anti-S1 signal for all 20 vaccinated saliva samples are above the break line of 1.004 (>cutoff value of 0.921 for anti-S1 IgG signal) on Y-axis. Anti-NCP signal for all 20 vaccinated saliva samples are below the break line of 1.004 (=cutoff value for anti-NCP IgG signal) on Y-axis. Therefore, 20 vaccinated saliva samples are all tested positive (100%) by nLF for anti-S1 IgG and all tested negative (0%) for anti-NCP IgG. b, Anti-S1 IgG and anti-NCP IgG signals for 21 infected samples (18 samples were collected 3-144 days post symptom onset, and 3 samples had no information regarding the number of days between symptom onset to sample collection). Anti-S1 and anti-NCP signals for all 21 infected saliva samples are above the break line of 1.004 on Y-axis, indicating all 21 COVID-19 infected saliva samples are tested positive for both anti-S1 IgG (100%) and anti-NCP IgG (100%).

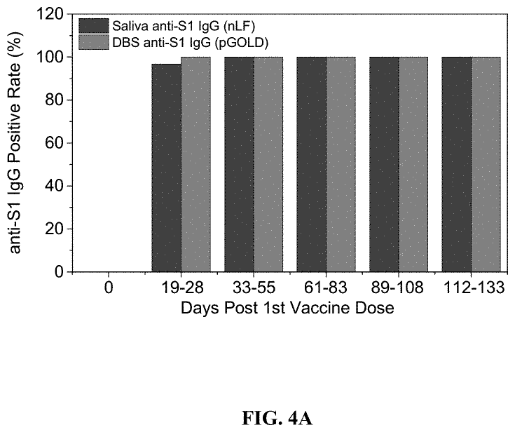

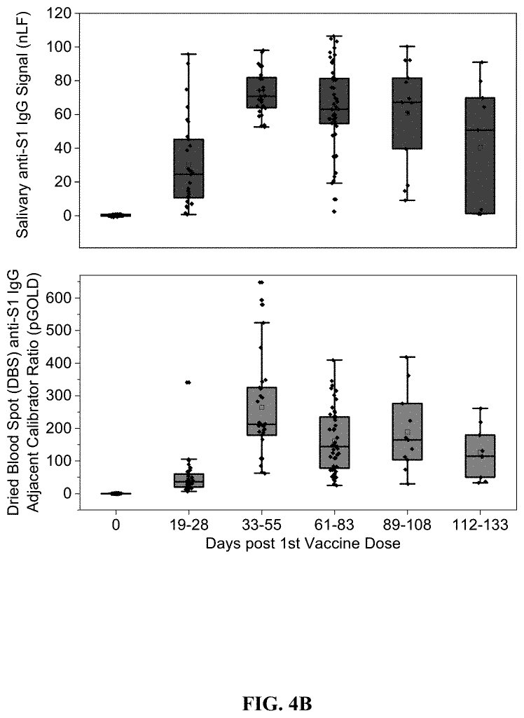

FIGS. 4A and 4B show Comparison of salivary antibody measured by nLF assay and antibodies in blood measured by pGOLD™ assay. (a) Anti-S1 IgG positive rate. Based on sample collection time after the 1st dose of vaccines, the samples were divided into 6 groups: D0 (before the 1st dose of vaccines), D19-28 (19-28 days, right before the 2nd dose of vaccines), D33-55 (33-55 days), D61-83 (61-83 days), D89-108 (89-108 days), and D112-133 (112-133 days). Salivary anti-S1 IgG positive rates tested using nLF in each group were 0% (DO), 96.55% (D19-28), 100% (D33-55), 100% (D61-83), 100% (D89-108) and 100% (D112-133), respectively. The anti-S1 IgG positive rates for dried blood spot (DBS) samples tested using pGOLD™ assay were 0% for DO samples and 100% for all of the vaccinated groups. b, Salivary anti-S1 IgG detected by nLF assay and DBS anti-S1 IgG detected by pGOLD™ assay. Anti-S1 IgG in both saliva and DBS were detected with >96.5% positivity within 19-28 days after the initial dose of mRNA vaccines, and further increased to higher levels after the second vaccine dose. The anti-S1 IgG levels reached a maximum at day 33-55 and remained elevated with slight decline from the peak during the follow up timepoints (day 61-83, 89-108, and 112-133).

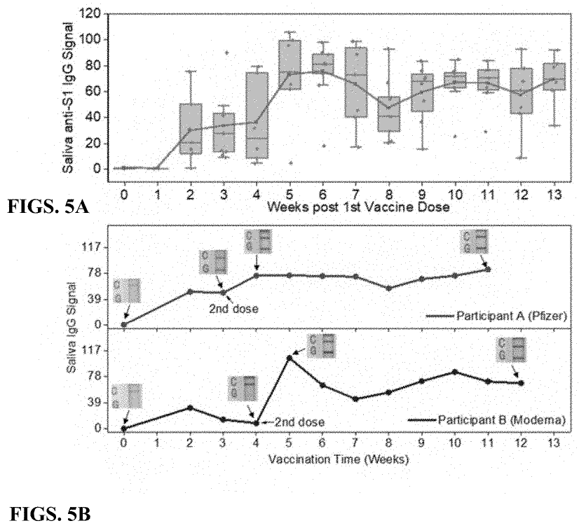

FIGS. 5A and 5B show Salivary antibody level monitoring post vaccination for individuals. a, a, Salivary anti-S1 IgG level monitoring post vaccination for 9 participants (7 from Pfizer, 2 from Moderna). Saliva samples are binned by weeks (1 or 2 weeks intervals), and their anti-S1 IgG levels are recorded up to 13 weeks using our nLF assay. b, Salivary anti-S1 IgG level changes over 13 weeks post vaccination of a representative participant who received Pfizer vaccine. c, Salivary anti-S1 IgG level changes over 13 weeks post vaccination of a representative participant who received Moderna vaccine.

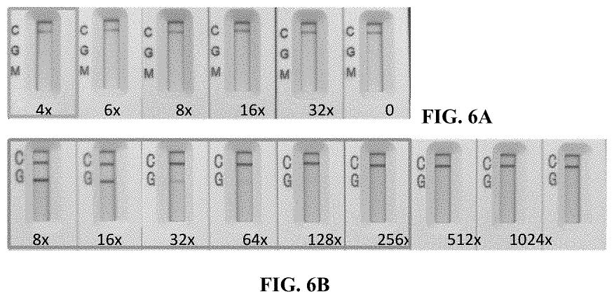

FIGS. 6A and 6B show Detection limit for anti-spike IgG in serum of nLF and MidaSpot. Purified recombinant anti-SARS-CoV-2 spike protein IgG (2.48 mg mL-1) was diluted into a negative serum sample and conducted serial dilutions (4× to 1024× dilutions) to determine the detection limit of nLF and MidaSpot. a, Detection limit for anti-spike IgG in serum of MidaSpot. Nirmidas' MidaSpot™ COVID-19 rapid Antibody Combo Detection Kit was approved by FDA EUA for POC testing. Based on the testing instruction, 10 μl serum sample was applied to the sample window followed by adding 4 drops (˜100 μl) of running buffer, and after 20 min the result was recorded. MidaSpot presents a positive signal for the 4× diluted sample and negative signals for more diluted serum samples, giving a detection limit of 620 μg mL-1.b, Detection limit for anti-spike IgG in serum of nLF. nLF testing followed the protocol described in the Examples below. The nLF is able to detect anti-spike IgG in the 256× diluted serum sample (9.69 μg mL-1 of IgG), presenting a ˜64 times lower detection limit of anti-spike IgG than that of the MidaSpot.

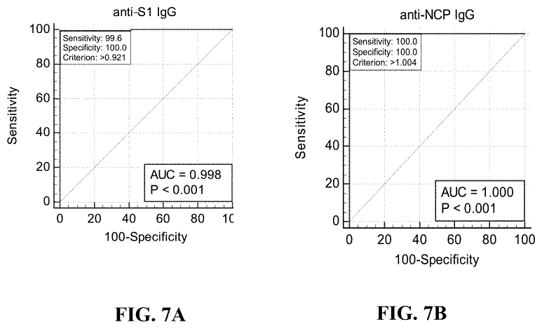

FIGS. 7A and 7B show ROC curves for the nLF SARS-CoV-2 IgG assay. a, ROC curves for the nLF SARS-CoV-2 anti-S1 IgG assay based on 34 negative, 21 saliva samples from PCR confirmed COVID-19 patients (18 samples were collected 3-144 days post symptom onset, and 3 samples had no information regarding the number of days between symptom onset to sample collection), and 206 vaccinated (mRNA vaccines from Pfizer and Moderna) saliva samples (collected ≥19 days post 1st vaccine shot) without prior COVID-19. b, ROC curves for the nLF SARS-CoV-2 anti-NCP IgG assay based on 12 negative and 21 PCR-positive COVID-19 saliva samples.

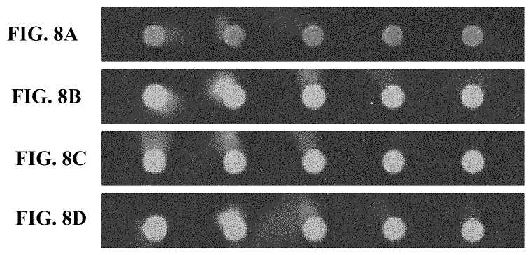

FIGS. 8A, 8B, 8C, and 8D show pGOLD™ results for representative DBS samples from group D19-28. a, pGOLD™ result for an anti-S1 IgG low positive DBS sample. b-d, pGOLD™ results for three representative anti-S1 IgG positive DBS samples.

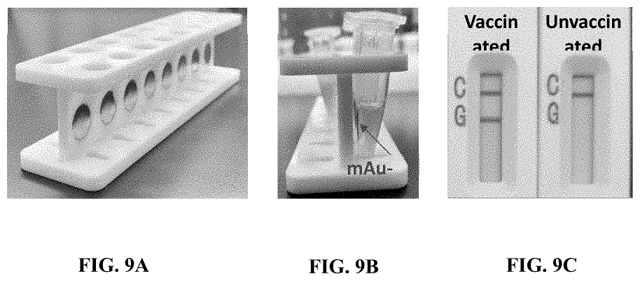

FIGS. 9A, 9B, and 9C show a magnetic rack for removing unbound non-specific antibodies. b, mAu-S1-IgG complex collection by magnetic force. c, Representative results from mAu based nLF for anti-S1 IgG positive and negative saliva samples.

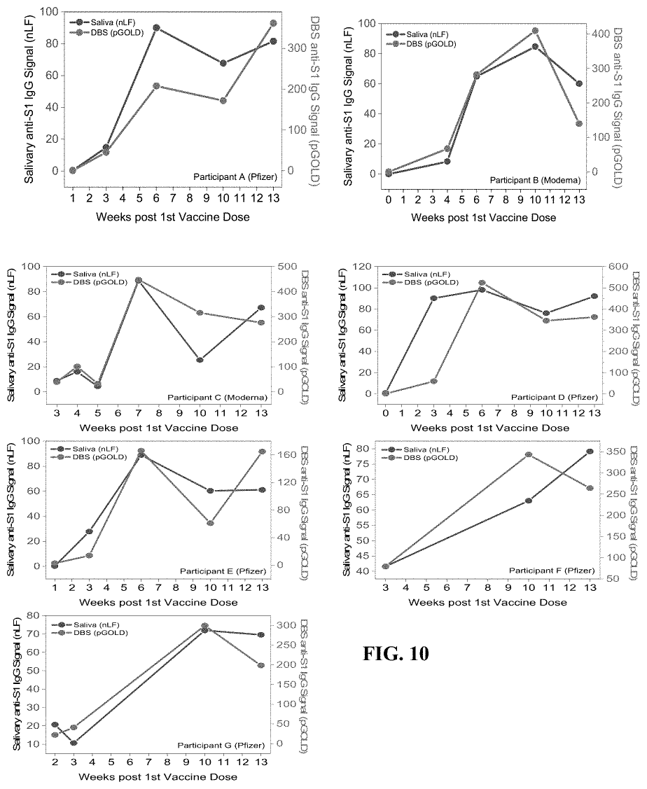

FIG. 10 shows Salivary anti-S1 IgG 1-13 weeks IgG post vaccination in different individuals.

### DETAILED DESCRIPTION

### Example 1

A New Lateral Flow (nLF) Assay Platform

The concentration of IgG antibodies in human saliva is much lower (˜15 μg mL⁻¹) than in serum or blood (˜15000 μg mL⁻¹). Antibodies against SARS-CoV-2 spike protein in saliva are more difficult to detect than in sera of COVID-19 infected individuals [11, 17, 20, 23]. Several lab-based assays have been developed to meet this challenge, but not for saliva based LF antibody tests.

For our nLF SARS-CoV-2 IgG antibody saliva test, we first prepared and optimized Au nanoparticle-antigen complexes. The Au nanoparticles were either pure gold (˜50 nm) or magnetic gold comprised an iron-oxide Fe₃O₄ core and an Au shell (see Method). When the Au-antigen (antigen=S1 or NCP of SARS-CoV-2) complexes were incubated with positive saliva samples, IgG antibodies specific to the viral antigen were captured to form Au-antigen-IgG complexes (FIG. 1a (i-iii)). Then a micro-centrifuging or magnetic purification step was applied to collect the Au-antigen-IgG complexes and remove the unbound, non-specific antibodies. The resulting Au-antigen-IgG complexes were dispersed in a running buffer and added to a specially designed LF testing card to flow through the signal and control lines (FIG. 1a (iv)). The card was comprised of a sample pad, a conjugate pad with pre-deposited colloidal gold nanoparticle-biotinylated BSA (Au-biotin-BSA) complexes, a test line immobilized with anti-human IgG, a control line immobilized with streptavidin, and a terminal absorbent pad (FIG. 1b). When antigen-specific IgG positive samples were used for the nLF, both test and control lines were detectable on the card, indicating a positive test, while negative samples or buffer alone only produced a red line in the control line (FIG. 1c). The step of removing unbound non-specific antibodies by micro-centrifuging or magnetic purification was critical to preventing competing antibodies in the sample from binding to the test line, drastically enhancing the detection sensitivity of specific antibodies over the traditional LF assay. The total nLF assay time was ˜25 min.

### Performance of the nLF Assay

We first compared the detection limit of nLF to that of a conventional LF test with FDA EUA (MidaSpot COVID-19 antibody combo developed by Nirmidas Biotech Inc.). We spiked purified recombinant anti-SARS-CoV-2 spike protein IgG (2.48 mg mL⁻¹) into a negative serum sample to various concentrations for determining the limit of detections by nLF and MidaSpot. MidaSpot showed positive signals for 4× and lower dilutions, giving a detection limit of 620 mL⁻¹ of antibody (Figure S1a). The nLF detected anti-spike IgG even in the 256× diluted sample (9.69 μg mL⁻¹), affording a ˜64 times lower detection limit than the conventional LF approach (Figure S1b) owing to the removal of excess, non-specific serum IgG by nLF.

We also spiked purified recombinant anti-SARS-CoV-2 spike protein IgG into a negative healthy saliva sample and performed serial dilutions for establishing the nLF assay detection limit for anti-spike IgG in saliva. By quantifying the signal using ImageJ software, we obtained an estimated antibody detection limit of ˜0.30 μg mL⁻¹, ˜50-fold lower than the total antibody concentration (˜15 μg mL⁻¹) in human saliva (FIG. 2a) [23].

We next focused on nLF assays of SARS-CoV-2 antibodies in saliva samples from vaccinated and COVID-19 infected cohorts. The major SARS-CoV-2 antigenic targets of human IgG are the spike protein and nucleocapsid (NCP). The SARS-CoV-2 spike protein is used as the target antigen and encoded by the mRNA of vaccines from Pfizer and Moderna. Successfully vaccinated individuals without prior COVID-19 by the two mRNA vaccines should only test positive for anti-S1 IgG but not for anti-NCP IgG [25-27]. COVID-19 infected patients are known to test positive for both anti-spike protein IgG and anti-NCP IgG [28]. The nLF assay platform is flexible for testing human IgG antibodies against any protein target by simply changing Au-antigen complexes while using the same test card. Using Au-S1 and Au-NCP complexes, we readily developed nLF tests for evaluating IgG antibodies against SARS-CoV-2 spike protein and NCP in saliva samples (FIG. 2b). With these tests, we observed a typical vaccinated saliva sample (42 days post 1st RNA vaccine dose from Moderna) exhibiting positive anti-S1 IgG and negative anti-NCP IgG, while an infected saliva sample (81 days post symptom onset) showing both positive anti-S1 IgG and anti-NCP IgG (FIG. 2c). (FIG. 2c). The same pattern was observed for large numbers of vaccinated (20) and infected samples (21) tested.

Salivary Antibody Patterns: Vaccination Vs. Infection

To fully establish the nLF assay for saliva anti-S1 IgG testing, we tested 34 unvaccinated healthy saliva samples without prior COVID-19 infection, 21 saliva samples from PCR confirmed COVID-19 patients (18 samples were collected 3-144 days post symptom onset, and 3 samples had no information regarding the number of days between symptom onset to sample collection), and 205 vaccinated (with mRNA vaccines from Pfizer and Moderna) saliva samples (collected ≥19 days post 1st vaccine shot from subjects without prior COVID-19 infection). For salivary SARS-CoV-2 anti-S1 IgG, a cut-off value of 0.921 T-line intensity unit (ImageJ) was determined via the receiver operator characteristic (ROC) curve (Figure S2a). The nLF saliva assay afforded 100% specificity without false positive for the 34 unvaccinated COVID-19 free cohort. Salivary SARS-CoV-2 anti-S1 IgG antibodies were detected in 21 out of 21 COVID-19 infected subjects (100%), and in 204 out 205 vaccinated subjects (99.51%). For the 205 vaccinated subjects, only 1 saliva sample on day 21 post the 1st vaccine dose was tested negative.

For the anti-NCP IgG nLF assay, a cut-off value was determined by testing 12 unvaccinated healthy control saliva samples without prior COVID-19 and saliva from 21 previously COVID-19 infected individual. The ROC analysis gave a cutoff value of 1.004 intensity unit under the criteria of 100% specificity (Figure S2b). All of the 21 COVID-19 infected saliva samples were tested positive for both anti-S1 IgG (100%) and anti-NCP IgG (100%) (FIG. 3b). In contrast, with 20 vaccinated saliva samples tested by nLF on day 42-55 post 1st vaccine dose, 0 out of 20 (0%) was tested positive for anti-NCP IgG (FIG. 3a) while all 20 (100%) were tested positive for anti-S1 IgG. This result suggested that salivary anti-S1 and anti-NCP IgG testing with the nLF platform offers an effective and non-invasive approach to differentiating immune responses due to vaccination (for mRNA targeting spike protein) from prior COVID-19 infections.

### Salivary Antibody Measured by nLF Assay and Antibodies in Blood Measured by a Lab-Based Serology Assay

To further validate our nLF assay for monitoring SARS-CoV-2 IgG trend post vaccination, we compared nLF IgG saliva test results with dried blood spot (DBS) serology test results using a previously established quantitative lab-test, the pGOLD assay [11]. We longitudinally measured 64 previously COVID-19 free individuals who received mRNA vaccines (51 from Pfizer, 13 from Moderna) and donated matched saliva and DBS samples at multiple timepoints over ˜4 months. Samples were divided into 6 groups based on collection time after the 1^{st} vaccine dose: D0 (before the 1st dose of vaccines), D19-28 (19-28 days, right before the 2nd vaccine dose), and fully vaccinated groups D33-55 (33-55 days), D61-83 (61-83 days), D89-108 (89-108 days), and D112-133 (112-133 days). With nLF, salivary anti-S1 IgG positivity were 0% (0/20), 96.43% (27/28), 100% (30/30), 100% (49/49), 100% (14/14) and 100% (9/9) for the 6 groups respectively, corroborating with anti-S1 IgG positivity measured in matched DBS samples by the quantitative pGOLD assay (FIG. 4a). Only one vaccinated saliva sample from group D19-28 was not detected positive to anti-S1 IgG by the nLF assay, with its matched DBS sample (Figure S3a) also showing a much weaker signal than other samples (Figure S3b-d) in the same group by the pGOLD assay.

The evolution of salivary anti-S1 IgG and anti-S1 IgG levels in matched DBS samples were monitored by nLF and pGOLD respectively over ˜4 months post vaccination, showing similar trends (FIG. 4b). Specifically, anti-S1 IgG in both saliva and DBS showed >96.4% positivity on day 19-28 after the first dose of mRNA vaccines, and further increased to higher levels after the second vaccine dose. The anti-S1 IgG levels reached a maximum on day 33-55, then remained elevated with a slow declining trend from the peak during the follow up timepoints (day 61-83, 89-108, and 112-133). This trend was consistent with a recent report on antibody persistence over 6 months after the 2nd of the Moderna mRNA1273 vaccine [29]. One disadvantage of the nLF assay, when compared to NIR fluorescence based pGOLD, is the lower dynamic range of the test line intensity, making it less differentiating in the high end of antibody concentrations. Nevertheless, we observed a similar trend of anti-S1 IgG in saliva detected by nLF assay and anti-S1 IgG in DBS detected by pGOLD assay (FIG. 4), suggesting saliva-based assay using the nLF could represent a rapid, noninvasive alternative for SARS-CoV-2 IgG testing and longitudinal monitoring.

To explore personalized monitoring of SARS-CoV-2 antibody persistence, we followed several vaccinated participants closely (FIG. 5 and FIG. 10) and binned their saliva samples by weeks (1 or 2 weeks intervals), and recorded their salivary anti-S1 IgG levels up to 13 weeks using our nLF assay. The general trend observed was that the anti-S1 IgG levels of most participants rose 2 weeks after the 1st mRNA vaccine dose. After the 2nd dose at 3-4 weeks, the anti-S1 IgG levels were boosted further, peaking approximately in the 5-7 weeks period, and remained relatively high over ˜10 weeks with modest decline from the peak (FIG. 5 and FIG. 10). The antibody levels and details of evolution were highly variable among all participants. Importantly, the salivary and blood anti-S1 levels for individuals followed the same trend of evolution post vaccinations (FIG. 5a, 5b for two examples & FIG. 10 for 5 more examples), suggesting the feasibility of non-invasive salivary antibody measurements by nLF for longitudinal assessment of vaccination effects. The anti-S1 IgG of participant A (Pfizer vaccine) increased in week 2 and leveled in week 3. After the 2^{nd} vaccine dose in week 3, the anti-S1 IgG increased further in week 4 and stayed stable followed by a modest decline (FIG. 5a). For participant B, a moderate anti-S1 IgG was detected in week 2 post the 1^{st} vaccine dose (Moderna), and the IgG level declined gradually in week 3 and 4. After the 2nd vaccine dose in week 4, the IgG level jumped drastically in week 5, then declined later with some fluctuations during the following weeks (FIG. 5b).

### Discussion

A simple but important feature of the nLF platform for rapid antibody detection is the removal of abundant, non-specific antibodies from a sample to afford higher analytical sensitivity (by ˜50-fold over conventional LF) and signal linearity. This step can be done in POC settings using simple micro-centrifuging (handheld, battery operated one available) or magnetic separation by employing magnetic nanoparticles residing in the core of a gold shell and permanent magnets for rapid washing (FIG. 9). Although this is an additional step relative to conventional LF antibody test, it is crucial to obtain IgG signals on the test lines that reflect the specific antibody concentrations in the sample due to the elimination of competing antibodies that always lower the true signal.

The nLF approach is applicable to detecting antibodies in all major body fluids including serum, plasma, venous and capillary whole blood and dried blood spot, for assessing immune responses to a wide range of infectious diseases or vaccines in POC settings within 25 minutes. It is also useful for detecting autoantibodies for various autoimmune diseases. The rapid test is semi-quantitative especially with the integration of an LF reader that detects light scattering of gold nanoparticles. A wide range of fluorescent or luminescent particles can also be employed for the nLF platform, boosting the nLF assay dynamic range by orders of magnitude for biomarker quantification.

We presented findings of longitudinal testing of salivary and blood IgG antibodies for individuals at various timepoints post vaccination up to ˜4 months. The study will continue for >1 year to correlate salivary and blood antibody levels and their relations with immunity to COVID-19 infection. A limitation of our work is that rigorous immunity assessments require serological neutralizing antibody quantification, raising the question of relevance of anti-SARS-CoV-2 spike protein levels in saliva and blood. However, neutralization assays are time consuming and employ live viruses, unsuitable for mass vaccination studies. Several simplified neutralization assays have been developed without using live viruses. Importantly, there have been mounting evidence that neutralizing antibody levels in COVID-19 patients are well correlated with antibodies against spike protein on SARS-CoV-2 surface. Further, our results here showed that salivary anti-RBD were well correlated with that in the blood for the mRNA vaccinated cohort as a whole (FIG. 4) and at the individual level (FIG. 5 and FIG. 10). Such correlation has been seen recently for COVID-19 patients and in various other infectious diseases in the past few decades. All of these results present a compelling case for monitoring salivary anti-spike protein IgG in vaccinated population as a non-invasive approach to assessing antibody levels and immunity. We clearly observed similar evolution trends and patterns for anti-spike protein IgG in saliva and in blood. These antibodies are entirely resulted from vaccination, and when immunity wanes, salivary antibodies could also diminish.

### Conclusion

With >99.5% sensitivity for SARS-CoV-2 spike protein IgG detection in vaccinated saliva samples (19 days post first vaccine shot) at 100% specificity, the rapid nLF assay provides an efficient and easy tool to evaluate population immunity to COVID-19, identifying potential immunity gaps and susceptible populations to inform targeted vaccination/prevention strategies like a booster vaccine dose. The flexible designability of the nLF assay platform not only enables users to easily differentiate vaccination from infection, but also gives the potential to detect other infected and autoimmune diseases by adjusting the Au-detecting agent complexes. With further improvements and modifications of the nLF platform such as introducing magnetic particles and a LF test reader, the nLF assay will be more user friendly for POC and can be translated into home testing.

### EXAMPLES

### Biological Samples and Materials

21 PCR-confirmed COVID-19 saliva samples were provided by Sinai Hospital of Baltimore. One set of paired saliva and dried blood spot (DBS) samples of 57 vaccinated participants (46 from Pfizer, 11 from Moderna) at multiple timepoints post vaccination are collected at Sinai Hospital of Baltimore. Another set of paired saliva and DBS samples at multiple time points post vaccination are collected from 7 vaccinated participants (5 from Pfizer, 2 from Moderna) at Nirmidas Biotech Inc. 2 participants at Nirmidas Biotech Inc. donated saliva samples at multiple time points post vaccination.

A 10 μl blood sample by finger stick was collected and applied to DBS cards at determined timepoints and stored at −20° C. Paired saliva samples at the same timepoints were collected through a simple method involving spitting into a plastic tube and stored at −20° C.

SARS-CoV-2 spike (S1) (40591-V08H) was purchased from Sino Biological. SARS-CoV-2 nucleocapsid protein (NCP) was purchased from (??? information)

### Chemicals

All chemicals were purchased from Sigma-Aldrich, and were used as received without further purification, except where mentioned otherwise.

### Preparation of Detection Au-Antigen Complex.

The colloidal gold (Au) nanoparticle solution was prepared by adding 0.7 ml 1 wt. % sodium citrate solution to 80 ml boiling gold chloride solution (0.0125 wt. %). After 30 min of reaction, the colloidal Au nanoparticles were prepared and was cooled to room temperature for Au-antigen preparation. The magnetic Au (mAu) nanoparticle containing a Fe3O4 core and an Au shell is a Nirmidas product. For Au-antigen/mAu-antigen complex preparation, 10 ug of S1 or NCP was mixed with 1.25 ml colloidal Au/mAu solution for 30 min at room temperature, leading to the ionic adsorption of SARS-CoV-2 antigens on the surface of the colloidal Au or mAu particles. Followed by blocking with 1% bovine serum albumin (BSA) in borate buffer (20 mM boric acid, 0.1% PEG20000, pH 9.0) for 30 min and StartingBlock (37538, Thermo Fisher Scientific) for 30 min. After blocking processes, the mixture was centrifuged at 10000 rpm for 5 min. The supernatant was discarded and the pellet containing Au-antigen/mAu-antigen complex was suspended in 60 μL Tris buffer (20 mM Tris, 5% Trehalose, 0.3% casein, 0.05% Tween20, pH 8.0), and stored at −20° C. for later usage.

### Fabrication of Immunochromatographic Strip

The immunochromatographic strip was composed of five components, a plastic backing, a sample pad, a conjugate pad, a nitrocellulose membrane and an absorbent pad. The sample pads and the conjugate pads were treated with 20 mM Tris buffer containing blocking agent and dried at 37° C. and 25% relative humidity. The Mouse anti human IgG (2 mg/ml) or the streptavidin (1 mg/ml) in PBS (1% wt. trehalose) was dispensed at the test or the control line on the nitrocellulose membrane, using a HM3030XYZ dispenser (Shanghai KinbioTech Co., Ltd., China) at a rate of 1.0 μL/cm and then dried at 37° C. The gold nanoparticles conjugated with biotin-BSA were applied to the treated conjugate pad and then dried completely. The absorption pad, nitrocellulose membrane, pretreated conjugate pad, and sample pad were attached to a plastic backing and assembled as a strip with a 1.5 mm overlap, sequentially. The assembled plate was cut into 4-mm-wide pieces, using an automatic strip cutter ZQ2000 (Shanghai Kinbio Tech Co., Ltd., China). The generated strip products were packaged in a plastic bag with desiccant and stored at room temperature.

### nLF Assay

For saliva sample, 200 μl saliva was mixed with 10 μl Au-S1 and 50 μl Tris buffer in a 1.5 ml minicentrifuge tube. For serum sample, 5 μl saliva was mixed with 10 μl Au-S1 and 390 Tris buffer in a 1.5 ml minicentrifuge tube. After a 10 min incubation at room temperature, 1 ml Tris buffer was added to the tube and the mixture was centrifuged with a mini centrifuge for 5 min. The supernatant was discarded, and the pellet was suspended in 100 μL running buffer (1×PBS, 0.05% Tween20, 1M NaCl) to add to the assay card. The results were read after 5 min of migration. The test line image intensity and background intensity were read by ImageJ (downloadable at https://imagej.net/Welcome). The test line signal of nLF was determined by the test line image intensity subtracted by background intensity.

pGOLD™ Antibody Assay

A 2 mm circle was punched out from each DBS (comprised of 10 μl dried finger stick whole blood sample) and dissolved in 160 μL sample buffer (10% FBS, 0.05% Tween20 and 0.1% NaN3 in 1×PBS) under shaking for 1 h at room temperature. After centrifugation at 3000 g for 5 min, the DBS-dissolved sample solution was heat inactivated at 56° C. for 30 minutes. The heat-inactivated solution was used for pGOLD assay immediately or stored at 4° C. for later usage. The pGOLD antibody assay was performed in a 64-well format as described in our previous work [11]. 1) Blocking: All wells were blocked with a blocking buffer containing 2% BSA in 1×PBS for 30 min at room temperature; 2) Sample incubation: each well was then incubated with 100 μL of 2× diluted DBS-dissolved sample solution in 10% FBST (10% FBS including 0.05% Tween20) for 1 h at room temperature. A mixture of a dilute IgG, IgM positive sample as signal normalizer was loaded into the neighboring/adjacent well on the same row for each sample tested, and a blank control (10% FBST only) was included on each biochip; 3) Secondary antibody incubation: each well was subsequently incubated with a mixture of 4 nM iFluor™820-labeled anti-human IgG secondary antibody, 4 nM CF647-labeled anti-human IgM secondary antibody, and 4 nM iFluor™820-labeled streptavidin in a 2% BSA solution for 30 minutes at room temperature. Each well was washed six times with 1×PBST (1×PBS with 0.05% Tween20) between steps. The iFluor™820-labeled streptavidin in the detection step binds to BSA-biotin spots in each well on the pGOLD™ biochip, and the signal was used as an “intrawell signal normalizer”. In parallel, row-dependent, antigen-specific “adjacent well” normalization was performed to normalize the variations antigen printing/slide variation on each slide. Anti-S1 IgG levels were calculated/defined as the ratio of anti-S1 signal of the sample to that of the adjacent well normalizer and multiplied by 100.

### Embodiments

In some examples, including any of the foregoing, set forth herein is a method for performing a lateral flow (LF) test for antibody detection in a body fluid, the method comprising: (a) conjugating antigens to particles to form particle-antigen complexes; (b) incubating the particle-antigen complexes with a body fluid sample to bind antibodies in the body fluid specific to the antigen, thereby producing particle-antigen-antibody-complexes in solution with the body fluid; (c) removing non-specific unbound antibodies from the particle-antigen-antibody-complexes in solution with the body fluid, resulting in the particle-antigen-antibody complexes being in a solution without non-specific unbound antibodies; (d) contacting the particle-antigen-antibody complexes in solution without non-specific unbound antibodies to the sample well of a LF test card to flow through a test line and a control line in a LF strip; (e) detecting and quantifying bound particle-antigen-antibody complexes on the test line and control line; (f) determining the concentration of antibody in the body fluid based on the test line signals or ratio between test line signal/control line signal; and (g) assessing immune responses due to a disease or vaccination based on the antibody concentration.

In some examples, including any of the foregoing, the antigen is from an infectious disease agent selected from Acute Flaccid Myelitis (AFM); Anaplasmosis; Anthrax; Babesiosis; Botulism; Brucellosis; Campylobacteriosis; Carbapenem-resistant Infection (CRE/CRPA); Chancroid; Chikungunya Virus Infection (Chikungunya); Chlamydia; Ciguatera (Harmful Algae Blooms (HABs); Clostridium Difficile Infection; Clostridium Perfringens (Epsilon Toxin); Coccidioidomycosis fungal infection (Valley fever); COVID-19 (Coronavirus Disease 2019); COVID-19 (Coronavirus Disease 2019); Creutzfeldt-Jacob Disease, transmissible spongiform encephalopathy (CJD); Cryptosporidiosis (Crypto); Cyclosporiasis; Dengue, 1,2,3,4 (Dengue Fever); Diphtheria; E. coli infection, Shiga toxin-producing (STEC); Eastern Equine Encephalitis (EEE); Ebola Hemorrhagic Fever (Ebola); Ehrlichiosis; Encephalitis, Arboviral or parainfectious; Enterovirus Infection, Non-Polio (Non-Polio Enterovirus); Enterovirus Infection, D68 (EV-D68); Giardiasis (Giardia); Glanders; Gonococcal Infection (Gonorrhea); Granuloma inguinale; Haemophilus Influenza disease, Type B (Hib or H-flu); Hantavirus Pulmonary Syndrome (HPS); Hemolytic Uremic Syndrome (HUS); Hepatitis A (Hep A); Hepatitis B (Hep B); Hepatitis C (Hep C); Hepatitis D (Hep D); Hepatitis E (Hep E); Herpes; Herpes Zoster, zoster VZV (Shingles); Histoplasmosis infection (Histoplasmosis); Human Immunodeficiency Virus/AIDS (HIV/AIDS); Human Papillomavirus (HPV); Influenza (Flu); Lead Poisoning; Legionellosis (Legionnaires Disease); Leprosy (Hansens Disease); Leptospirosis; Listeriosis (Listeria); Lyme Disease; Lymphogranuloma venereum infection (LGV); Malaria; Measles; Melioidosis; Meningitis, Viral (Meningitis, viral); Meningococcal Disease, Bacterial (Meningitis, bacterial); Middle East Respiratory Syndrome Coronavirus (MERS-CoV); Multisystem Inflammatory Syndrome in Children (MIS-C); Mumps; Norovirus; Paralytic Shellfish Poisoning; Pediculosis (Lice, Head and Body Lice); Pelvic Inflammatory Disease (PID); Pertussis (Whooping Cough); Plague; Bubonic, Septicemic, Pneumonic (Plague); Pneumococcal Disease (Pneumonia); Poliomyelitis (Polio); Powassan; Psittacosis (Parrot Fever); Pthiriasis (Crabs; Pubic Lice Infestation); Pustular Rash diseases (Small pox, monkeypox, cowpox); Q-Fever; Rabies; Ricin Poisoning; Rickettsiosis (Rocky Mountain Spotted Fever); Rubella, Including congenital (German Measles); Salmonellosis gastroenteritis (Salmonella); Scabies Infestation (Scabies); Scombroid; Septic Shock (Sepsis); Severe Acute Respiratory Syndrome (SARS); Shigellosis gastroenteritis (Shigella); Smallpox; Staphyloccal Infection, Methicillin-resistant (MRSA); Staphylococcal Food Poisoning, Enterotoxin-B Poisoning (Staph Food Poisoning); Staphylococcal Infection, Vancomycin Intermediate (VISA); Staphylococcal Infection, Vancomycin Resistant (VRSA); Streptococcal Disease, Group A (invasive) (Strep A (invasive)); Streptococcal Disease, Group B (Strep-B); Streptococcal Toxic-Shock Syndrome, STSS, Toxic Shock (STSS, TSS); Syphilis, primary, secondary, early latent, late latent, congenital; Tetanus Infection, tetani (Lock Jaw); Trichomoniasis (Trichomonas infection); Trichonosis Infection (Trichinosis); Tuberculosis (TB); Tuberculosis (Latent); Tularemia (Rabbit fever); Typhoid Fever, Group D; Typhus; bacterial Vaginosis; Vaping-Associated Lung Injury; Varicella (Chickenpox); Vibrio cholerae; Vibriosis; Viral Hemorrhagic Fever; West Nile Virus; Yellow Fever; Yersenia (Yersinia); and Zika Virus.

In some examples, including any of the foregoing, the antigen is associated with an autoinmmune disease selected from the group consisting of: Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticarial; Axonal & neuronal neuropathy (AMAN); Baló disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum; Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inversa); Hypogammalglobulinemia; IgA Nephropathy; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB; Multiple sclerosis; Myasthenia gravis; Myelin Oligodendrocyte Glycoprotein Antibody Disorder; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (PCD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, III; Polymyalgia rheumatica; Polymyositis; Postmyocardial infarction syndrome; Postpericardiotomy syndrome; Primary Biliary Cholangitis; Primary sclerosing cholangitis; Progesterone dermatitis; Psoriasis; Psoriatic arthritis; Pure red cell aplasia (PRCA); Pyoderma gangrenosum; Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Thyroid eye disease (TED); Tolosa-Hunt syndrome (THS); Transverse myelitis; Type 1 diabetes; Ulcerative colitis (UC); Undifferentiated connective tissue disease (UCTD); Uveitis; Vasculitis; Vitiligo; and Vogt-Koyanagi-Harada Disease.

In some examples, including any of the foregoing, the particle is selected from a nanoparticle and a microparticle.

In some examples, including any of the foregoing, the particle is a member selected from the group consisting of metallic gold particle, a polymeric particle, a fluorescent particle, a rare-earth down-converting particle, a rare-earth up-converting particle, an europium (Eu) containing particle, a visually brightly colored particle, and a magnetic particle, a black colored particle, a black carbon particle, a Se particle.

In some examples, including any of the foregoing, the particle has a characteristic dimension equal to, or between 10-500 nm.

In some examples, including any of the foregoing, the particle has core-shell structures of at least two different compositions.

In some examples, including any of the foregoing, the core-shell structures are selected from iron oxide/gold, quantum dot/inorganic layer, rare-earth/inorganic layer, rare-earth/gold, polymer/gold, and combinations thereof.

In some examples, including any of the foregoing, the particle fluoresces visible, near-infrared (700-100 nm) light or short wave infrared (SWIR) (1000-1700 nm emission) light.

In some examples, including any of the foregoing, the particle is a polymer bead comprising an organic dye that fluoresces visible or near-infrared (700-100 nm emission) light or SWIR (1000-1700 nm emission) light.

In some examples, including any of the foregoing, the particle is inorganic and is bonded to an inorganic dye that fluoresces visible or near-infrared (700-100 nm emission) light or SWIR (1000-1700 nm emission) light.

In some examples, including any of the foregoing, the body fluid is a member selected from the group consisting of whole venous blood, capillary blood, serum, plasma, saliva, sputum, oral swab and urine.

In some examples, including any of the foregoing, the process includes removing non-specific unbound antibody from the particle-antigen-antibody-complexes in solution with the body fluid by centrifugation to aggregate the particle-antigen-antibody complexes as a precipitate leaving the non-specific unbound antibodies and body fluid as a supernatant, discarding the supernatant, and resuspending the precipitated particle-antigen-antibody complexes.

In some examples, including any of the foregoing, the removing the non-specific unbound antibody from the particle-antigen-antibody-complexes in solution with the body fluid is by magnetic separation to aggregate the particle-antigen-antibody complexes, leaving the non-specific unbound antibodies and body fluid as a supernatant, discarding the supernatant, and resuspending the aggregated particle-antigen-antibody complexes.

In some examples, including any of the foregoing, the removing the non-specific unbound antibody from the particle-antigen-antibody-complexes in solution with the body fluid sample is by filtration to retain the particle-antigen-antibody complexes and remove the non-specific unbound free antibodies.

In some examples, including any of the foregoing, the removing the non-specific unbound antibody from the particle-antigen-antibody-complexes in solution with the body fluid is by microfluidics to separate particle-antigen-antibody complexes and non-specific unbound antibodies by size.

In some examples, including any of the foregoing, the removing non-specific unbound antibody from the particle-antigen-antibody-complexes in solution with the body fluid is by passing through a chromatography column to separate particle-antigen-antibody complexes and non-specific unbound antibodies by size.

In some examples, including any of the foregoing, the LF test card comprises a plastic backing, a sample well with a sample pad, a conjugate pad with pre-deposited nanoparticle-biotinylated protein (such as BSA), a nitrocellulose membrane containing a test line with printed anti human IgG and a control line with printed streptavidin.

In some examples, including any of the foregoing, the detecting and quantifying bound particle-antigen-antibody complexes on the test line is by visual examination to determine the presence or absence of captured particle-antigen-antibody complexes, and deciding the positive or negative status of the antibody of interest in the body fluid.

In some examples, including any of the foregoing, the detecting and quantifying bound particle-antigen-antibody complexes on the test line is by a portable reader that detects and quantifies light scattering by the particle-antigen-antibody complexes captured on the test line.

In some examples, including any of the foregoing, the detecting and quantifying bound particle-antigen-antibody complexes on the test line is by taking a photo of the LF strip using a smart phone and analyzing the test line brightness using a phone APP and converting to antibody concentration.

In some examples, including any of the foregoing, the detecting and quantifying bound particle-antigen-antibody complexes on the test line is by using a portable reader that measures visible or NIR or SWIR fluorescence emission of the particle-antigen-antibody-complexes captured on the test line and converting the measured visible or NIR or SWIR fluorescence emission to antibody concentration in the body fluid.

In some examples, including any of foregoing, set forth herein is a method for detecting antigen specific IgG antibodies in a population of IgG antibodies, comprising: (i) contacting a saliva sample, from a subject, comprising a population of IgG antibodies, with gold nanoparticles (Au nanoparticle) having antigens bonded thereto, wherein the antigens specifically bind complementary IgG antibodies in the saliva sample; (ii) forming Au nanoparticle-antigen-IgG complexes in solution with the saliva; (iii) separating the Au nanoparticle-antigen-IgG complexes to provide separated Au nanoparticle-antigen-IgG complexes; and (iv) detecting the separated Au nanoparticle-antigen-IgG complexes by contacting the separated Au nanoparticle-antigen-IgG complexes to a lateral flow (LF) test card, wherein the LF test card comprises: a plastic backing, a sample pad, a conjugate pad comprising gold nanoparticles coated with-biotinylated bovine serum albumin (Au-biotin-BSA) complexes, a nitrocellulose membrane a test line that comprises anti-human IgG antibodies, a control line comprising streptavidin, and an absorbent pad; wherein the contacting is on the sample pad, and the separated Au nanoparticle-antigen-IgG complexes flow from the sample pad to the absorbent pad; wherein a signal at the test line and the control line indicates the presence of antigen specific IgG antibodies in the population of IgG antibodies.

In some examples, including any of the foregoing, the Au nanoparticle size is 10-500 nm.

In some examples, including any of the foregoing, the Au nanoparticle has core-shell structures of at least two different compositions.

In some examples, including any of the foregoing, the core-shell structures are selected from iron oxide/gold, rare-earth/gold, polymer/gold, and combinations thereof.

In some examples, including any of the foregoing, the Au nanoparticle fluoresces visible, near-infrared (700-100 nm) light or short wave infrared SWIR (1000-1700 nm emission) light.

In some examples, including any of the foregoing, the Au-nanoparticle comprises a polymer bead having visible, near-infrared (700-100 nm emission), or SWIR (1000-1700 nm emission) fluorescent organic dyes bonded thereto.

In some examples, including any of the foregoing, the Au nanoparticle is bonded with an inorganic fluorescent dye that fluoresces visible, near-infrared (700-100 nm) or light or short wave infrared SWIR (1000-1700 nm emission) light.

In some examples, including any of the foregoing, the Au nanoparticle is bonded with an organic fluorescent dye that fluoresces visible, near-infrared (700-100 nm) or light or short wave infrared SWIR (1000-1700 nm emission) light.

In some examples, including any of the foregoing, the separating is by centrifugation to aggregate the Au nanoparticle-antigen-IgG complexes as a precipitate, leaving the non-specific unbound IgG antibodies in solution with saliva as a supernatant, discarding the supernatant, and resuspending the precipitated Au nanoparticle-antigen-IgG complexes.

In some examples, including any of the foregoing, the separating is by magnetic separation to aggregate the Au nanoparticle-antigen-IgG complexes, leaving the non-specific unbound IgG antibodies in solution with saliva as a supernatant, discarding the supernatant, and resuspending the aggregated Au nanoparticle-antigen-IgG complexes.

In some examples, including any of the foregoing, the separating is by filtration to retain the Au nanoparticle-antigen-IgG complexes and remove the non-specific unbound IgG antibodies.

In some examples, including any of the foregoing, the separating is by microfluidics to separate Au nanoparticle-antigen-IgG complexes and non-specific unbound IgG antibodies by size.

In some examples, including any of the foregoing, the separating is by passing through a chromatography column to separate Au nanoparticle-antigen-IgG complexes and non-specific unbound IgG antibodies by size.

In some examples, including any of the foregoing, the detecting the separated Au nanoparticle-antigen-IgG complexes on the test line is by visual examination to determine the presence or absence of a signal at the test line.

In some examples, including any of the foregoing, the detecting the separated Au nanoparticle-antigen-IgG complexes on the test line is by a portable reader determines the presence or absence of a signal at the test line by detecting and quantifying light scattering by the Au nanoparticle-antigen-IgG complexes.

In some examples, including any of the foregoing, the detecting the separated Au nanoparticle-antigen-IgG complexes on the test line is by taking a photo of the of the test line on the LF slide using a smart phone and analyzing the test line brightness using a phone APP and converting to antibody concentration.

In some examples, including any of the foregoing, the detecting the separated Au nanoparticle-antigen-IgG complexes on the test line is by using a portable reader that measures visible or NIR or SWIR fluorescence emission at the test line and converting the measured visible or NIR or SWIR fluorescence emission to antibody concentration in the saliva.

In some examples, including any of the foregoing, set forth herein is a method of identifying a subject, in need thereof, as having been mRNA vaccinated against SARS-CoV-2 or as having been infected by SARS-CoV-2, comprising (i) contacting a saliva sample, from the subject, comprising a population of IgG antibodies with gold nanoparticles (Au nanoparticle) having SARS-CoV-2 antigens bonded thereto, wherein the antigens specifically bind complementary IgG antibodies in the saliva sample, if present, and wherein the antigens are selected from SARS-CoV-2 nucleocapsid protein (NCP) and SARS-CoV-2 spike protein (51); (ii) forming Au nanoparticle-SARS-CoV-2-antigen-IgG complexes; (iii) separating the Au nanoparticle-SARS-CoV-2-antigen-IgG complexes to provide separated Au nanoparticle-SARS-CoV-2-antigen-IgG complexes; and (iv) detecting the Au nanoparticle-SARS-CoV-2-antigen-IgG complexes by contacting the separated Au nanoparticle-SARS-CoV-2-antigen-IgG complexes to a new lateral flow (nLF) test slide, wherein the nLF test slide comprises: a plastic backing, a sample pad, a conjugate pad comprising gold nanoparticles coated with-biotinylated bovine serum albumin (Au-biotin-BSA) complexes, a nitrocellulose membrane a test line that comprises anti-human IgG antibodies, a control line comprising streptavidin, and an absorbent pad; wherein the contacting is on the sample pad, and the separated Au nanoparticle-antigen-IgG complexes flow from the sample pad to the absorbent pad; and (v) wherein a detection of both NCP and S1 indicates that subject was infected with SARS-CoV-2; and (vi) treating the subject with a compound or other therapy to mitigate symptoms, prevent spread of SARS-CoV-2 infection, facilitate therapy for others, and/or improve symptoms of COVID-19.

In some examples, including any of the foregoing, the Au nanoparticle size is 10 nm-500 nm.

In some examples, including any of the foregoing, the Au nanoparticle has core-shell structures of at least two different compositions.

In some examples, including any of the foregoing, the core-shell structures are selected from iron oxide/gold, rare-earth/gold, polymer/gold, and combinations thereof.

In some examples, including any of the foregoing, the Au nanoparticle fluoresces visible, near-infrared (700 nm-100 nm) light or short wave infrared SWIR (1000 nm-1700 nm emission) light.

In some examples, including any of the foregoing, the Au-nanoparticle comprises a polymer bead having visible, near-infrared (700-100 nm emission), or SWIR (1000 nm-1700 nm emission) fluorescent organic dyes bonded thereto.

In some examples, including any of the foregoing, the Au nanoparticle is bonded with an inorganic fluorescent dye that fluoresces visible, near-infrared (700 nm-100 nm) or light or short wave infrared SWIR (1000 nm-1700 nm emission) light.

In some examples, including any of the foregoing, the Au nanoparticle is bonded with an organic fluorescent dye that fluoresces visible, near-infrared (700 nm-100 nm) or light or short wave infrared SWIR (1000 nm-1700 nm emission) light.

In some examples, including any of the foregoing, the separating is by centrifugation to aggregate the Au nanoparticle-antigen-IgG complexes as a precipitate, leaving the non-specific unbound IgG antibodies in solution with saliva as a supernatant, discarding the supernatant, and resuspending the precipitated Au nanoparticle-antigen-IgG complexes.

In some examples, including any of the foregoing, the separating is by magnetic separation to aggregate the Au nanoparticle-antigen-IgG complexes, leaving the non-specific unbound IgG antibodies in solution with saliva as a supernatant, discarding the supernatant, and resuspending the aggregated Au nanoparticle-antigen-IgG complexes.

In some examples, including any of the foregoing, the separating is by filtration to retain the Au nanoparticle-antigen-IgG complexes and remove the non-specific unbound IgG antibodies.

In some examples, including any of the foregoing, the separating is by microfluidics to separate Au nanoparticle-antigen-IgG complexes and non-specific unbound IgG antibodies by size.

In some examples, including any of the foregoing, the method includes separating by passing through a chromatography column to separate Au nanoparticle-antigen-IgG complexes and non-specific unbound IgG antibodies by size.

In some examples, including any of the foregoing, the method includes detecting the separated Au nanoparticle-antigen-IgG complexes on the test line is by visual examination to determine the presence or absence of a signal at the test line.

In some examples, including any of the foregoing, the method includes detecting the separated Au nanoparticle-antigen-IgG complexes on the test line is by a portable reader determines the presence or absence of a signal at the test line by detecting and quantifying light scattering by the Au nanoparticle-antigen-IgG complexes.

In some examples, including any of the foregoing, the method includes detecting the separated Au nanoparticle-antigen-IgG complexes on the test line is by taking a photo of the of the test line on the LF slide using a smart phone and analyzing the test line brightness using a phone APP and converting to antibody concentration.

In some examples, including any of the foregoing, the method includes detecting the separated Au nanoparticle-antigen-IgG complexes on the test line is by using a portable reader that measures visible or NIR or SWIR fluorescence emission at the test line and converting the measured visible or NIR or SWIR fluorescence emission to antibody concentration in the saliva.

In some examples, including any of the foregoing, set forth herein is kit for implementing any of the methods set forth herein.

### Compounds and Therapies

The methods disclosed herein can be a factor in determining if a patient should be treated for COVID-19 or not, or can be taken into consideration when deciding what follow-up tests should be done, defining the response to therapies, monitoring any possible recurrences of COVID-19, and identifying new therapeutic targets.

A subject diagnosed as having COVID-19 or as having been infected with SARS-CoV-2 may be treated by any known method in the art.

In some embodiments, a subject diagnosed as having COVID-19 is treated by administering therapeutically effective amount of Remdesivir (e.g. Veklury® see e.g., Beigel J H, et al. (2020) N Engl J Med. 383(19):1813-1826) or a compound such as that disclosed in U.S. Pat. Nos. 10,905,698, 10,987,329, or 10,980,756.

In other embodiments, a subject diagnosed as having COVID-19 is treated by administering therapeutically effective amount of an anti-SARS-CoV-2 antibody cocktail such as REGEN-COV™ (casirivimab with imdevimab). In some embodiments, REGEN-COV™ is administered early in a SARS-CoV-2 infection. In other embodiments, a subject diagnosed as having COVID-19 is treated with palliative therapy.

In some embodiments, a subject with past infection who has immunity e.g., a subject having high avidity SARS-CoV-2 antibodies, may be advised that they can safely work in essential settings such as health care, public safety and the service industry. They also can be advised that they can safely work in “non-essential” settings with less need for extreme personal protection.

Alternatively, a subject with past infection who has immunity and high avidity SARS-CoV-2 specific antibodies, can be advised to donate convalescent plasma which can be used to treat other individuals who are experiencing COVID-19, thus preventing severe COVID complications for those other individuals.

Because the methods disclosed herein can detect very early infection (through the ability to accurately and specifically identify the IgM) a person having asymptomatic or pre-symptomatic infection can be advised to seek early treatment e.g., by administration of REGEN-COV™, and/or to engage in immediate isolation or quarantine to prevent spread.

Furthermore, symptomatic individuals can be notified if their symptoms are COVID-related and if not COVID, then they can be advised what other virus they might have and appropriate care for the symptoms can be recommended.

### Kits

In one aspect, provided herein are kits useful for diagnosing or prognosticating SARS-CoV-2 infection and/or COVID-19 in a subject. Kits typically comprise fluorescence signal enhancing plasmonic gold slides, spotted with bound SARS-CoV-2 antigens, dye-labeled detection antibodies, standard controls, diluents, denaturing agents, and instructions for use.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

The following may provide additional background information:
[1] U. Ravens-Sieberer, A. Kaman, M. Erhart, J. Devine, R. Schlack, C. Otto, Impact of the COVID-19 pandemic on quality of life and mental health in children and adolescents in Germany, Eur Child Adolesc Psychiatry (2021).
[2] A. Fontanet, B. Autran, B. Lina, M. P. Kieny, S. S. A. Karim, D. Sridhar, SARS-CoV-2 variants and ending the COVID-19 pandemic, The Lancet 397(10278) (2021) 952-954.
[3] S. J. Salyer, J. Maeda, S. Sembuche, Y. Kebede, A. Tshangela, M. Moussif, C. Ihekweazu, N. Mayet, E. Abate, A. O. Ouma, J. Nkengasong, The first and second waves of the COVID-19 pandemic in Africa: a cross-sectional study, The Lancet 397(10281) (2021) 1265-1275.
[4] R. Padhan, K. P. Prabheesh, The economics of COVID-19 pandemic: A survey, Econ Anal Policy 70 (2021) 220-237.
[5] D. Skegg, P. Gluckman, G. Boulton, H. Hackmann, S. S. A. Karim, P. Piot, C. Woopen, Future scenarios for the COVID-19 pandemic, The Lancet 397(10276) (2021) 777-778.
[6] T. Tanaka, S. Okamoto, Increase in suicide following an initial decline during the COVID-19 pandemic in Japan, Nat Hum Behav 5(2) (2021) 229-238.
[7] S. Loomba, A. de Figueiredo, S. J. Piatek, K. de Graaf, H. J. Larson, Measuring the impact of COVID-19 vaccine misinformation on vaccination intent in the UK and USA, Nat Hum Behav 5(3) (2021) 337-348.
[8] J. Keehner, L. E. Horton, M. A. Pfeffer, C. A. Longhurst, R. T. Schooley, J. S. Currier, S. R. Abeles, F. J. Torriani, SARS-CoV-2 Infection after Vaccination in Health Care Workers in California, N Engl J Med 384(18) (2021) 1774-1775.
[9] M. S. Edward P. K. Parker, Beate Kampmann, Keeping track of the SARS-CoV-2 vaccine pipeline, Nature Reviews Immunology 20(650) (2020) 1.
[10] J. Van Elslande, E. Houben, M. Depypere, A. Brackenier, S. Desmet, E. Andre, M. Van Ranst, K. Lagrou, P. Vermeersch, Diagnostic performance of seven rapid IgG/IgM antibody tests and the Euroimmun IgA/IgG ELISA in COVID-19 patients, Clin Microbiol Infect 26(8) (2020) 1082-1087.
[11] T. Liu, J. Hsiung, S. Zhao, J. Kost, D. Sreedhar, C. V. Hanson, K. Olson, D. Keare, S. T. Chang, K. P. Bliden, P. A. Gurbel, U. S. Tantry, J. Roche, C. Press, J. Boggs, J. P. Rodriguez-Soto, J. G. Montoya, M. Tang, H. Dai, Quantification of antibody avidities and accurate detection of SARS-CoV-2 antibodies in serum and saliva on plasmonic substrates, Nat Biomed Eng 4(12) (2020) 1188-1196.
[12] X. F. Cai, J. Chen, J. Li Hu, Q. X. Long, H. J. Deng, P. Liu, K. Fan, P. Liao, B. Z. Liu, G. C. Wu, Y. K. Chen, Z. J. Li, K. Wang, X. L. Zhang, W. G. Tian, J. L. Xiang, H. X. Du, J. Wang, Y. Hu, N. Tang, Y. Lin, J. H. Ren, L. Y. Huang, J. Wei, C. Y. Gan, Y. M. Chen, Q. Z. Gao, A. M. Chen, C. L. He, D. X. Wang, P. Hu, F. C. Zhou, A. L. Huang, D. Q. Wang, A Peptide-Based Magnetic Chemiluminescence Enzyme Immunoassay for Serological Diagnosis of Coronavirus Disease 2019, J Infect Dis 222(2) (2020) 189-193.
[13] J. L. Wu, W. P. Tseng, C. H. Lin, T. F. Lee, M. Y. Chung, C. H. Huang, S. Y. Chen, P. R. Hsueh, S. C. Chen, Four point-of-care lateral flow immunoassays for diagnosis of COVID-19 and for assessing dynamics of antibody responses to SARS-CoV-2, J Infect 81(3) (2020) 435-442.
[14] F. A. Ania Wajnberg, Adolfo Firpo, Deena R. Altman, Mark J. Bailey, Mayce Mansour, Meagan McMahon, Philip Meade, Damodara Rao Mendu, Kimberly Muellers, Daniel Stadlbauer, Kimberly Stone, Shirin Strohmeier, Viviana Simon, Judith Aberg, David L. Reich, Florian Krammer, Carlos Cordon-Cardo, Robust neutralizing antibodies to SARS-CoV-2 infection persist for months, Science 370(6521) (2020) 4.
[15] K. K.-W. To, O. T.-Y. Tsang, W.-S. Leung, A. R. Tam, T.-C. Wu, D. C. Lung, C. C.-Y. Yip, J.-P. Cai, J. M.-C. Chan, T. S.-H. Chik, D. P.-L. Lau, C. Y.-C. Choi, L.-L. Chen, W.-M. Chan, K.-H. Chan, J. D. Ip, A. C.-K. Ng, R. W.-S. Poon, C.-T. Luo, V. C.-C. Cheng, J. F.-W. Chan, I. F.-N. Hung, Z. Chen, H. Chen, K.-Y. Yuen, Temporal profiles of viral load in posterior oropharyngeal saliva samples and serum antibody responses during infection by SARS-CoV-2: an observational cohort study, The Lancet Infectious Diseases 20(5) (2020) 565-574.
[16] M. A. MacMullan, A. Ibrayeva, K. Trettner, L. Deming, S. Das, F. Tran, J. R. Moreno, J. G. Casian, P. Chellamuthu, J. Kraft, K. Kozak, F. E. Turner, V. I. Slepnev, L. M. Le Page, ELISA detection of SARS-CoV-2 antibodies in saliva, Sci Rep 10(1) (2020) 20818.
[17] N. Pisanic, P. R. Randad, K. Kruczynski, Y. C. Manabe, D. L. Thomas, A. Pekosz, S. L. Klein, M. J. Betenbaugh, W. A. Clarke, O. Laeyendecker, P. P. Caturegli, H. B. Larman, B. Detrick, J. K. Fairley, A. C. Sherman, N. Rouphael, S. Edupuganti, D. A. Granger, S. W. Granger, M. H. Collins, C. D. Heaney, COVID-19 Serology at Population Scale: SARS-CoV-2-Specific Antibody Responses in Saliva, J Clin Microbiol 59(1) (2020).
[18] D. Ter-Ovanesyan, T. Gilboa, R. Lazarovits, A. Rosenthal, X. Yu, J. Z. Li, G. M. Church, D. R. Walt, Ultrasensitive Measurement of Both SARS-CoV-2 RNA and Antibodies from Saliva, Anal Chem 93(13) (2021) 5365-5370.
[19] P. Hettegger, J. Huber, K. Passecker, R. Soldo, U. Kegler, C. Nohammer, A. Weinhausel, High similarity of IgG antibody profiles in blood and saliva opens opportunities for saliva based serology, PLoS One 14(6) (2019) e0218456.
[20] K. T. A. Baweleta Isho, Michelle Zuo, Alainna J. Jamal, Bhavisha Rathod, Jenny H. Wang, Zhijie Li, Gary Chao, Olga L. Rojas, Yeo Myong Bang, Annie Pu, Natasha Christie-Holmes, Christian Gervais, Derek Ceccarelli, Payman Samavarchi-Tehrani, Furkan Guvenc, Patrick Budylowski, Angel Li, Aimee Paterson, Yue Feng Yun, Lina M. Marin, Lauren Caldwell, Jeffrey L. Wrana, Karen Colwill, Frank Sicheri, Samira Mubareka, Scott D. Gray-Owen, Steven J. Drews, Walter L. Siqueira, Miriam Barrios-Rodiles, Mario Ostrowski, James M. Rini, Yves Durocher, Allison J. McGeer, Jennifer L. Gommerman and Anne-Claude Gingras, Persistence of serum and saliva antibody responses to SARS-CoV-2 spike antigens in COVID-19 patients, Science Immunology 5(52) (2020).
[21] B. Isho, K. T. Abe, M. Zuo, A. J. Jamal, B. Rathod, J. H. Wang, Z. Li, G. Chao, O. L. Rojas, Y. M. Bang, A. Pu, N. Christie-Holmes, C. Gervais, D. Ceccarelli, P. Samavarchi-Tehrani, F. Guvenc, P. Budylowski, A. Li, A. Paterson, Y. F. Yun, L. M. Marin, L. Caldwell, J. L. Wrana, K. Colwill, F. Sicheri, S. Mubareka, S. D. Gray-Owen, S. J. Drews, W. L. Siqueira, M. Barrios-Rodiles, M. Ostrowski, J. M. Rini, Y. Durocher, A. J. McGeer, J. L. Gommerman, A.-C. Gingras, Evidence for sustained mucosal and systemic antibody responses to SARSCoV-2 antigens in COVID-19 patients, medRxiv (2020).
[22] A. Chakravarti, M. Matlani, M. Jain, Immunodiagnosis of dengue virus infection using saliva, Curr Microbiol 55(6) (2007) 461-4.
[23] T. J. Ketas, D. Chaturbhuj, V. M. Cruz-Portillo, E. Francomano, E. Golden, S. Chandrasekhar, G. Debnath, R. Diaz-Tapia, A. Yasmeen, W. Leconet, Z. Zhao, P. J. M. Brouwer, M. M. Cushing, R. W. Sanders, A. Cupo, P. J. Klasse, S. C. Formenti, J. P. Moore, Antibody responses to SARS-CoV-2 mRNA vaccines are detectable in saliva, bioRxiv (2021).
[24] K. M. Koczula, A. Gallotta, Lateral flow assays, Essays Biochem 60(1) (2016) 111-20.
[25] J. E. Ebinger, J. Fert-Bober, I. Printsev, M. Wu, N. Sun, J. C. Prostko, E. C. Frias, J. L. Stewart, J. E. Van Eyk, J. G. Braun, S. Cheng, K. Sobhani, Antibody responses to the BNT162b2 mRNA vaccine in individuals previously infected with SARS-CoV-2, Nat Med (2021).
[26] U. Sahin, A. Muik, E. Derhovanessian, I. Vogler, L. M. Kranz, M. Vormehr, A. Baum, K. Pascal, J. Quandt, D. Maurus, S. Brachtendorf, V. Lorks, J. Sikorski, R. Hilker, D. Becker, A. K. Eller, J. Grutzner, C. Boesler, C. Rosenbaum, M. C. Kuhnle, U. Luxemburger, A. Kemmer-Bruck, D. Langer, M. Bexon, S. Bolte, K. Kariko, T. Palanche, B. Fischer, A. Schultz, P. Y. Shi, C. Fontes-Garfias, J. L. Perez, K. A. Swanson, J. Loschko, I. L. Scully, M. Cutler, W. Kalina, C. A. Kyratsous, D. Cooper, P. R. Dormitzer, K. U. Jansen, O. Tureci, COVID-19 vaccine BNT162b1 elicits human antibody and TH1 T cell responses, Nature 586(7830) (2020) 594-599.
[27] Z. Wang, F. Schmidt, Y. Weisblum, F. Muecksch, C. O. Barnes, S. Finkin, D. Schaefer-Babajew, M. Cipolla, C. Gaebler, J. A. Lieberman, T. Y. Oliveira, Z. Yang, M. E. Abernathy, K. E. Huey-Tubman, A. Hurley, M. Turroja, K. A. West, K. Gordon, K. G. Millard, V. Ramos, J. Da Silva, J. Xu, R. A. Colbert, R. Patel, J. Dizon, C. Unson-O'Brien, I. Shimeliovich, A. Gazumyan, M. Caskey, P. J. Bjorkman, R. Casellas, T. Hatziioannou, P. D. Bieniasz, M. C. Nussenzweig, mRNA vaccine-elicited antibodies to SARS-CoV-2 and circulating variants, Nature 592(7855) (2021) 616-622.
[28] A. Stromer, R. Rose, O. Grobe, F. Neumann, H. Fickenscher, T. Lorentz, A. Krumbholz, Kinetics of Nucleo- and Spike Protein-Specific Immunoglobulin G and of Virus-Neutralizing Antibodies after SARS-CoV-2 Infection, Microorganisms 8(10) (2020).
[29] M. S. S. Nicole Doria-Rose, Mat Makowski, Sarah O'Connell, Adrian B. McDermott, Britta Flach, Julie E. Ledgerwood, John R. Mascola, Barney S. Graham, Bob C. Lin, Sijy O'Dell, Stephen D. Schmidt, Alicia T. Widge, Venkata-Viswanadh Edara, Evan J. Anderson, Lilin Lai, Katharine Floyd, Nadine G. Rouphael, Veronika Zarnitsyna, Paul C. Roberts, Mamodikoe Makhene, Wendy Buchanan, Catherine J. Luke, John H. Beigel, Lisa A. Jackson, Kathleen M. Neuzil, Hamilton Bennett, Brett Leav, Jim Albert, Pratap Kunwar, Antibody persistence through 6 months after the second dose of mRNA-1273 vaccine for Covid-19, The New England Journal of Medicine (2021) 3.

1. A method for performing a lateral flow (LF) test for antibody detection in a body fluid, the method comprising:
(a) conjugating antigens to particles to form particle-antigen complexes;
(b) incubating the particle-antigen complexes with a body fluid sample to bind antibodies in the body fluid specific to the antigen, thereby producing particle-antigen-antibody-complexes in solution with the body fluid;
(c) removing non-specific unbound antibodies from the particle-antigen-antibody-complexes in solution with the body fluid, resulting in the particle-antigen-antibody complexes being in a solution without non-specific unbound antibodies;
(d) contacting the particle-antigen-antibody complexes in solution without non-specific unbound antibodies to the sample well of a LF test card to flow through a test line and a control line in a LF strip;
(e) detecting and quantifying bound particle-antigen-antibody complexes on the test line and control line;
(f) determining the concentration of antibody in the body fluid based on the test line signals or ratio between test line signal/control line signal; and
(g) assessing immune responses due to a disease or vaccination based on the antibody concentration.

2. The method of claim 1, wherein the antigen is from an infectious disease agent selected from Acute Flaccid Myelitis (AFM); Anaplasmosis; Anthrax; Babesiosis; Botulism; Brucellosis; Campylobacteriosis; Carbapenem-resistant Infection (CRE/CRPA); Chancroid; Chikungunya Virus Infection (Chikungunya); Chlamydia; Ciguatera (Harmful Algae Blooms (HABs); Clostridium Difficile Infection; Clostridium Perfringens (Epsilon Toxin); Coccidioidomycosis fungal infection (Valley fever); COVID-19 (Coronavirus Disease 2019); COVID-19 (Coronavirus Disease 2019); Creutzfeldt-Jacob Disease, transmissible spongiform encephalopathy (CJD); Cryptosporidiosis (Crypto); Cyclosporiasis; Dengue, 1,2,3,4 (Dengue Fever); Diphtheria; E. coli infection, Shiga toxin-producing (STEC); Eastern Equine Encephalitis (EEE); Ebola Hemorrhagic Fever (Ebola); Ehrlichiosis; Encephalitis, Arboviral or parainfectious; Enterovirus Infection, Non-Polio (Non-Polio Enterovirus); Enterovirus Infection, D68 (EV-D68); Giardiasis (Giardia); Glanders; Gonococcal Infection (Gonorrhea); Granuloma inguinale; Haemophilus Influenza disease, Type B (Hib or H-flu); Hantavirus Pulmonary Syndrome (HPS); Hemolytic Uremic Syndrome (HUS); Hepatitis A (Hep A); Hepatitis B (Hep B); Hepatitis C (Hep C); Hepatitis D (Hep D); Hepatitis E (Hep E); Herpes; Herpes Zoster, zoster VZV (Shingles); Histoplasmosis infection (Histoplasmosis); Human Immunodeficiency Virus/AIDS (HIV/AIDS); Human Papillomavirus (HPV); Influenza (Flu); Lead Poisoning; Legionellosis (Legionnaires Disease); Leprosy (Hansens Disease); Leptospirosis; Listeriosis (Listeria); Lyme Disease; Lymphogranuloma venereum infection (LGV); Malaria; Measles; Melioidosis; Meningitis, Viral (Meningitis, viral); Meningococcal Disease, Bacterial (Meningitis, bacterial); Middle East Respiratory Syndrome Coronavirus (MERS-CoV); Multisystem Inflammatory Syndrome in Children (MIS-C); Mumps; Norovirus; Paralytic Shellfish Poisoning; Pediculosis (Lice, Head and Body Lice); Pelvic Inflammatory Disease (PID); Pertussis (Whooping Cough); Plague; Bubonic, Septicemic, Pneumonic (Plague); Pneumococcal Disease (Pneumonia); Poliomyelitis (Polio); Powassan; Psittacosis (Parrot Fever); Pthiriasis (Crabs; Pubic Lice Infestation); Pustular Rash diseases (Small pox, monkeypox, cowpox); Q-Fever; Rabies; Ricin Poisoning; Rickettsiosis (Rocky Mountain Spotted Fever); Rubella, Including congenital (German Measles); Salmonellosis gastroenteritis (Salmonella); Scabies Infestation (Scabies); Scombroid; Septic Shock (Sepsis); Severe Acute Respiratory Syndrome (SARS); Shigellosis gastroenteritis (Shigella); Smallpox; Staphyloccal Infection, Methicillin-resistant (MRSA); Staphylococcal Food Poisoning, Enterotoxin-B Poisoning (Staph Food Poisoning); Staphylococcal Infection, Vancomycin Intermediate (VISA); Staphylococcal Infection, Vancomycin Resistant (VRSA); Streptococcal Disease, Group A (invasive) (Strep A (invasive)); Streptococcal Disease, Group B (Strep-B); Streptococcal Toxic-Shock Syndrome, STSS, Toxic Shock (STSS, TSS); Syphilis, primary, secondary, early latent, late latent, congenital; Tetanus Infection, tetani (Lock Jaw); Trichomoniasis (Trichomonas infection); Trichonosis Infection (Trichinosis); Tuberculosis (TB); Tuberculosis (Latent); Tularemia (Rabbit fever); Typhoid Fever, Group D; Typhus; bacterial Vaginosis; Vaping-Associated Lung Injury; Varicella (Chickenpox); Vibrio cholerae; Vibriosis; Viral Hemorrhagic Fever; West Nile Virus; Yellow Fever; Yersenia (Yersinia); and Zika Virus.

3. The method of claim 1, wherein the antigen is associated with an autoinmmune disease selected from the group consisting of: Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticarial; Axonal & neuronal neuropathy (AMAN); Baló disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum; Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inversa); Hypogammalglobulinemia; IgA Nephropathy; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB; Multiple sclerosis; Myasthenia gravis; Myelin Oligodendrocyte Glycoprotein Antibody Disorder; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (PCD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, III; Polymyalgia rheumatica; Polymyositis; Postmyocardial infarction syndrome; Postpericardiotomy syndrome; Primary Biliary Cholangitis; Primary sclerosing cholangitis; Progesterone dermatitis; Psoriasis; Psoriatic arthritis; Pure red cell aplasia (PRCA); Pyoderma gangrenosum; Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Thyroid eye disease (TED); Tolosa-Hunt syndrome (THS); Transverse myelitis; Type 1 diabetes; Ulcerative colitis (UC); Undifferentiated connective tissue disease (UCTD); Uveitis; Vasculitis; Vitiligo; and Vogt-Koyanagi-Harada Disease.

4. The method of claim 1, wherein the particle is selected from a nanoparticle and a microparticle.

5. The method of claim 1, wherein the particle is a member selected from the group consisting of metallic gold particle, a polymeric particle, a fluorescent particle, a rare-earth down-converting particle, a rare-earth up-converting particle, an europium (Eu) containing particle, a visually brightly colored particle, and a magnetic particle, a black colored particle, a black carbon particle, a Se particle.

6. The method of claim 1, wherein the particle has a characteristic dimension equal to, or between 10-500 nm.

7. The method of claim 1, wherein the particle has core-shell structures of at least two different compositions.

8. The method of claim 7, wherein the core-shell structures are selected from iron oxide/gold, quantum dot/inorganic layer, rare-earth/inorganic layer, rare-earth/gold, polymer/gold, and combinations thereof.

9. The method of claim 1, wherein the particle fluoresces visible, near-infrared (700-100 nm) light or short wave infrared (SWIR) (1000-1700 nm emission) light.

10. The method of claim 1, wherein the particle is a polymer bead comprising an organic dye that fluoresces visible or near-infrared (700-100 nm emission) light or SWIR (1000-1700 nm emission) light.

11. The method of claim 1, wherein the particle is inorganic and is bonded to an inorganic dye that fluoresces visible or near-infrared (700-100 nm emission) light or SWIR (1000-1700 nm emission) light.

12. The method of claim 1, wherein the body fluid is a member selected from the group consisting of whole venous blood, capillary blood, serum, plasma, saliva, sputum, oral swab and urine.

13. The method of claim 1, wherein removing non-specific unbound antibody from the particle-antigen-antibody-complexes in solution with the body fluid is by centrifugation to aggregate the particle-antigen-antibody complexes as a precipitate leaving the non-specific unbound antibodies and body fluid as a supernatant, discarding the supernatant, and resuspending the precipitated particle-antigen-antibody complexes.

14. The method of claim 1, wherein removing non-specific unbound antibody from the particle-antigen-antibody-complexes in solution with the body fluid is by magnetic separation to aggregate the particle-antigen-antibody complexes, leaving the non-specific unbound antibodies and body fluid as a supernatant, discarding the supernatant, and resuspending the aggregated particle-antigen-antibody complexes.

15. The method of claim 1, wherein removing non-specific unbound antibody from the particle-antigen-antibody-complexes in solution with the body fluid sample is by filtration to retain the particle-antigen-antibody complexes and remove the non-specific unbound free antibodies.

16.-18. (canceled)

19. The method of claim 1, wherein detecting and quantifying bound particle-antigen-antibody complexes on the test line is by visual examination to determine the presence or absence of captured particle-antigen-antibody complexes, and deciding the positive or negative status of the antibody of interest in the body fluid.

20. The method of claim 1, wherein detecting and quantifying bound particle-antigen-antibody complexes on the test line is by a portable reader that detects and quantifies light scattering by the particle-antigen-antibody complexes captured on the test line.

21. (canceled)

22. The method of claim 1, wherein detecting and quantifying bound particle-antigen-antibody complexes on the test line is by using a portable reader that measures visible or NIR or SWIR fluorescence emission of the particle-antigen-antibody-complexes captured on the test line and converting the measured visible or NIR or SWIR fluorescence emission to antibody concentration in the body fluid.

23. A method for detecting antigen specific IgG antibodies in a population of IgG antibodies, comprising:
(i) contacting a saliva sample, from a subject, comprising a population of IgG antibodies, with gold nanoparticles (Au nanoparticle) having antigens bonded thereto, wherein the antigens specifically bind complementary IgG antibodies in the saliva sample;
(ii) forming Au nanoparticle-antigen-IgG complexes in solution with the saliva;
(iii) separating the Au nanoparticle-antigen-IgG complexes to provide separated Au nanoparticle-antigen-IgG complexes; and
(iv) detecting the separated Au nanoparticle-antigen-IgG complexes by contacting the separated Au nanoparticle-antigen-IgG complexes to a lateral flow (LF) test card, wherein the LF test card comprises:
a plastic backing,
a sample pad,
a conjugate pad comprising gold nanoparticles coated with-biotinylated bovine serum albumin (Au-biotin-BSA) complexes,
a nitrocellulose membrane
a test line that comprises anti-human IgG antibodies,
a control line comprising streptavidin, and
an absorbent pad;
wherein the contacting is on the sample pad, and the separated Au nanoparticle-antigen-IgG complexes flow from the sample pad to the absorbent pad;
wherein a signal at the test line and the control line indicates the presence of antigen specific IgG antibodies in the population of IgG antibodies.

24.-39. (canceled)

40. A method of identifying a subject, in need thereof, as having been mRNA vaccinated against SARS-CoV-2 or as having been infected by SARS-CoV-2, comprising
(i) contacting a saliva sample, from the subject, comprising a population of IgG antibodies with gold nanoparticles (Au nanoparticle) having SARS-CoV-2 antigens bonded thereto,
wherein the antigens specifically bind complementary IgG antibodies in the saliva sample, if present, and wherein the antigens are selected from SARS-CoV-2 nucleocapsid protein (NCP) and SARS-CoV-2 spike protein (S1);
(ii) forming Au nanoparticle-SARS-CoV-2-antigen-IgG complexes;
(iii) separating the Au nanoparticle-SARS-CoV-2-antigen-IgG complexes to provide separated Au nanoparticle-SARS-CoV-2-antigen-IgG complexes; and
(iv) detecting the Au nanoparticle-SARS-CoV-2-antigen-IgG complexes by contacting the separated Au nanoparticle-SARS-CoV-2-antigen-IgG complexes to a new lateral flow (nLF) test slide, wherein the nLF test slide comprises:
a plastic backing,
a sample pad,
a conjugate pad comprising gold nanoparticles coated with-biotinylated bovine serum albumin (Au-biotin-BSA) complexes,
a nitrocellulose membrane
a test line that comprises anti-human IgG antibodies,
a control line comprising streptavidin, and
an absorbent pad;
wherein the contacting is on the sample pad, and the separated Au nanoparticle-antigen-IgG complexes flow from the sample pad to the absorbent pad; and
(v) wherein a detection of both NCP and S1 indicates that subject was infected with SARS-CoV-2; and
(vi) treating the subject with a compound or other therapy to mitigate symptoms, prevent spread of SARS-CoV-2 infection, facilitate therapy for others, and/or improve symptoms of COVID-19.

41.-57. (canceled)
